(19) United States
(12) Patent Application Publication
(10) Pub. No.: US 2011/0020929 A1
(43) Pub. Date: Jan. 27, 2011

(54) PARTIALLY ACTIVE MICROFLUIDIC SYSTEM FOR 3D CELL CULTIVATION AND METHOD FOR PERFUSION THEREOF

(75) Inventors: Andreas Schober, Furth (DE); Caroline Augspurger, Ilmenau (DE); Frank Weise, Ilmenau (DE); Uta Fernekorn, Erfurt (DE); Christian Hildmann, Erfurt (DE); Jorg Hampl, Erfurt (DE)

Correspondence Address: MAYER & WILLIAMS PC, 251 NORTH AVENUE WEST, 2ND FLOOR, WESTFIELD, NJ, 07090, US

(73) Assignee: TECHNISCHE UNIVERSITAT ILMENAU, Ilmenau (DE)

(21) Appl. No.: 12/675,195

(22) PCT Filed: Apr. 15, 2009
(86) PCT No.: PCT/EP09/54444

§ 371 Date: Jul. 7, 2010

(30) Foreign Application Priority Data
Apr. 15, 2008 (DE) 102008019691.6

(51) Int. Cl. C12N 5/071; C12M 3/00

(52) U.S. Cl. 435/366; 435/305.2

(57) ABSTRACT

The invention relates to a partially active microfluidic system for cell cultivation comprising a multiwell system (4) with multiple wells (17) and cavities formed therein that hold cells. Each cavity (11) is formed by at least one inlay (3) comprising an opening (1) that does not open up directly into the cavity (11), but rather into a space (17) surrounding the cavity, wherein the medium present in the surrounding space communicates with the medium contained in the cavity (11) by way of a sieve structure in the inlay. The invention is suitable in particular for 3D cell cultivation.

The invention relates to parallel microfluidic systems, whose manufacture is cost advantageous, for the cultivation of advanced cell cultures (3D culture, stem cells, etc.) which can be used advantageously in any laboratory working in biotechnology and biomedicine, and which is moreover HTS capable (HTS: High Throughput Screening). Such systems are in principle appropriate for use in the search for active ingredients in ADME/Tox screenings, and their marketing potential is consequently high. ADME/Tox studies are used to examine substances for their properties in the (human) organism with regard to Absorption, Distribution, Metabolization, Excretion and Toxicity.

In numerous applications in medicine and pharmaceutical research, or in the cosmetic industry, the cultivation of biological materials is a crucial task. In this context, to the extent possible, one should culture the cell material, which can be tissue sections, for example, from biopsies or from primary cells that were collected from animals or humans, cell lines, or genetically modified cells, in such a way that their natural functional and living capacities are maintained, or the desired functions are implemented as optimally as possible.

The bandwidth of application here ranges from the preparation of test systems for pharmaceutical and cosmetic research to medical research (for example, stem cell research), and to the production of vaccines, and basic research. An important aspect here relates both to saving valuable cell material, and also to allowing many different parallel runs for testing different parameters.

In a tissue, the nutrients and substances for differentiation (for example, messenger substances) must be prepared in sufficient quantity. This cannot be ensured using diffusion alone in the case of tissues consisting of several cell layers (3D cell culture), rather active transport mechanisms, analogous to those in the blood circulation (and also the lymph system) must be used, by means of which the required substances can undergo intercellular introduction and removal. Here it has been found that the geometry, for example, of support systems, frameworks for cell colonization, the separation and the guidance of fluidic supply channels, as well as the density, the species and the type of the cell colonization constitute important parameters that are decisive in tissue engineering with regard to the successful cultivation or manufacture of tissues. In addition, the induction of differentiation processes by the targeted release of active ingredients in a tissue remains an unsolved problem which to date has been approached only via “trial and error” methods.

For the proper functioning of a tissue or organ, many parameters are of significance. To ensure the maintenance of the organ and tissue functions in vitro, it is not only necessary to reproduce the correct molecular architecture, but also above all the correct macroscopic architecture of the cell aggregation. For many applications, it is essential to culture the different cell species together (cocultivation), because one cell species produces substances that influence the other cell species, for example, in differentiation. In other application, the focus of interest has been on the interactions of the cell types and the targeted metabolization by the cell species in sequence. Moreover, it is known that primary cells frequently lose their cell type specific (differentiated) functions when they are cultured in a monolayer (single layer). Therefore, various efforts have been made to develop cell culture systems that better reproduce the three-dimensional in vivo situation of the corresponding tissue.

One possibility is the microfluidically supported cell cultivation of cells in 3D culture. Here, one starts with 3D cultures in microstructured supports (3D cell culture sample supports also referred to as 3D CellChips), which are already in the test stage, including for long-term cultivation. A 3D cell culture sample support (for example, Matrigrid) here denotes a two-dimensionally or three-dimensionally constructed support structure for the three-dimensional cultivation of cells, which are preferably perforated, thus allowing medium to flow through them.

Such a sample support (3D CellChip) is known, for example, from WO 93/07258, where the orders of magnitudes for the support framework in advanced cell culture are based on physiological parameters. The height of the support framework for cells in a three-dimensional cultivation which is supplied from both sides should not exceed 300 μm, if there is no active flow through the cell layer. The prototype of the described cell carrier accommodates, on a surface area of 1 cm², approximately 900 (30×30) microcontainers having the dimensions 300 μm×300 μm×300 μm (L×B×H) and a wall thickness of 50 μm. The bottom has pores that ensure unimpeded substance exchange already in case of superfusion (flow above or below), but also the flow of medium through the cell aggregation (perfusion).

The use of microsystem techniques for the manufacture of bioreactors has been found to be very advantageous for the defined cultivation of advanced cell cultures. The experimentally needed diversity with regard to different cell lines, serums, media, and active ingredients can be addressed economically only by the use of miniaturized systems.

From the dissertation by C. Augspurger (Leipzig 2007), it is known that such sample supports are operated in accordance with standards, in bioreactors with several 25 mL volumes using external pumps as self-enclosed systems. However, these bioreactors can be used only limitedly for some applications, including in pharmaceutical research, because they are not compatible with the interfaces of automated laboratory technology, such as, for example, with the microtiter plate format (or also “MTP footprint”). In addition, it is not possible to introduce test substances automatically in these bioreactors. Moreover, they are characterized by an insufficient capacity to allow parallel runs. An additional problem consists of the fact that the required biological material or the volume used is simply too large for many tasks for these bioreactors to be used advantageously for parallel runs.

For the desired miniaturization and parallelization on the basis of microtiter plates, the format and volumes are too large and expensive precisely for screening applications. Therefore, in different proposals, 3D structured polymers or polymer foams are introduced in 24- or 96-well microtiter plates as support structures (see also Wintermantel “Three-dimensional cell cultures;” TUM-Communications, 4-2006 (October 2006). However, these systems lack active perfusion. Active flow through the tissue and cell material is thus not possible. These systems therefore represent an unsatisfactory model for imitating organs in the living organism.

From U.S. Pat. No. 6,943,009 B2, a device for cell cultivation is known which has on its surface additional openings for the addition and the removal of solutions. However, the device has no integrated 3D structuring for a 3D cell culture with flow through it, and thus it is not suitable for cell cultivation that approximates in vivo conditions. In addition, in this system, only one mesh per well can be suspended in each case (the meshes cannot be stacked), which does not allow the cocultivation of different sequentially aligned cell types in the same well, but in different compartments.

US 2005/0191759 A1 shows a device and a method for carrying out a liquid phase microextraction. The apparatus comprises a fluid membrane and an optional carrier on a porous polymer substrate, which may be a hollow fiber.

In DE 602 16 076 T2, a hollow fiber membrane multiple container plate for enrichment and cleaning of samples is described, which has a plurality of containers for receiving several samples. Moreover, several hollow fibers are provided, of which in each case one is located in these containers, and in each case has a fluid extraction membrane that encloses an internal hollow cavity of the hollow fiber. The cultivation of cells and tissues in an in vivo situation can however not be achieved with this plate.

In general, known inlay systems present in part similar problems, for example, the above-mentioned proposal of Wintermantel et al., i.e., an active perfusion is not possible in such a microtiter plate, unless fluid is added or removed by means of a pipetting system. However, fundamental problems arise here. Due to the flow of fluid, the cells may become detached, i.e., the cell aggregate is capable of floating, and, above all, in this way it is impossible to carry out a circular throughflow. An additional problem of inlay systems is that, at the time of the introduction of precultured inlays that are colonized with cells, the risk of detachment of the cell aggregation as a result of the flow pressure during immersion also becomes relevant.

The problem of the present invention therefore is to overcome the disadvantages known from the state of the art, and, to develop, on the basis of conventional microtiter plates, a partially active microfluidic system for 2D and/or 3D cell cultivation for laboratory automation or for automated High Content Screening (HCS), i.e., the automatic determination of many biological and physical parameters, or the so-called High Throughput Screening (HTS). It is preferred for such a system to be also stackable (for example, for cocultivation).

This problem is solved by a partially active micro fluidic system according to the appended Claim 1, and by a method according to the appended dependent Claim 10.

A possibility for producing a partially active microfluidic system for 3D cell cultivation consists of the integration of membrane inlays, preferably as a two-dimensional or three-dimensional support structure for three-dimensional cultivation of cells, in a microtiter plate.

An advantageous embodiment of the system according to the invention is achieved by the integration of a suction system and of a collection compartment.

The invention is explained in greater detail below in reference to the drawing. In the drawing:

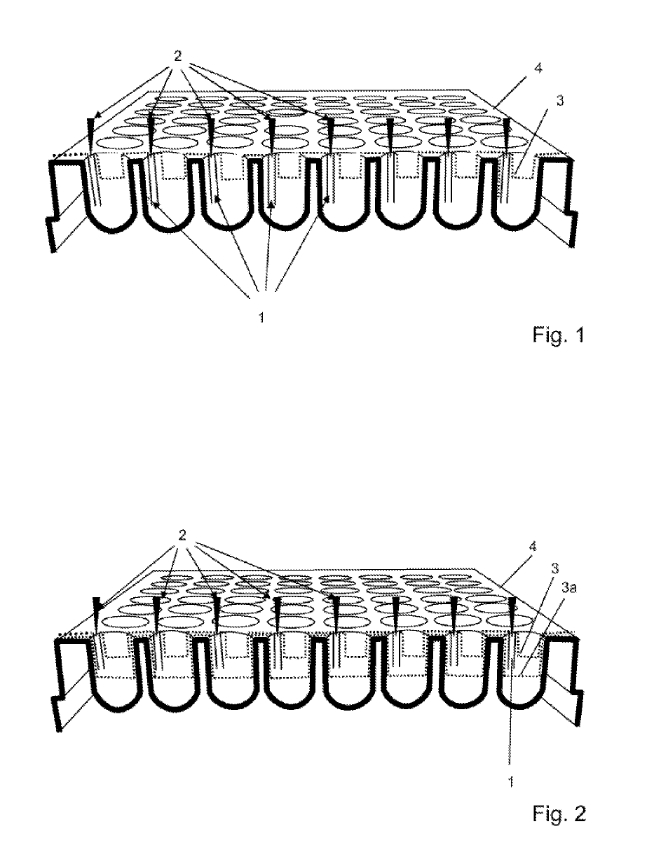

FIG. 1 shows a first embodiment according to the invention of a partially active microfluidic system for 3D cell cultivation;

FIG. 2 shows a second embodiment according to the invention of a partially active microfluidic system for 3D cell cultivation;

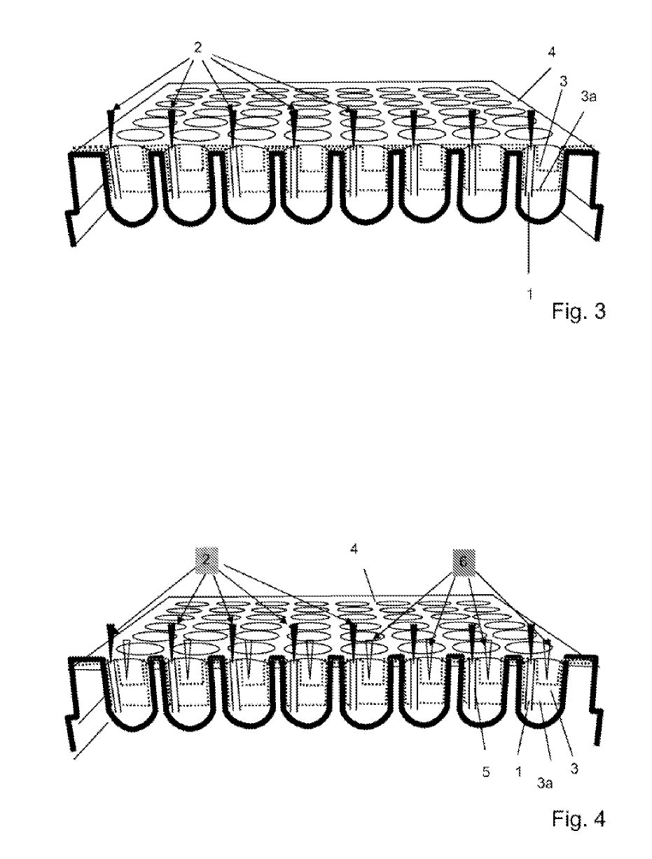

FIG. 3 shows a third embodiment according to the invention of a partially active microfluidic system for 3D cell cultivation;

FIG. 4 shows a representation of the principle of a method according to the invention for the perfusion of a partially active microfluidic system;

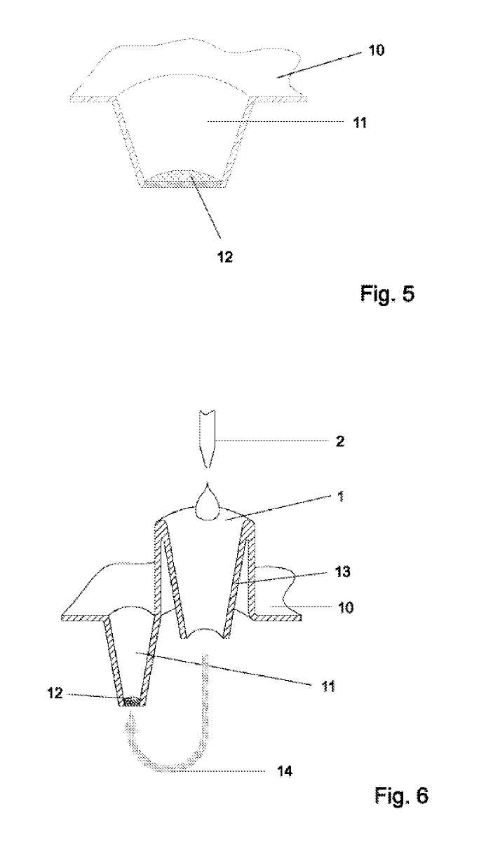

FIGS. 5-9 show detail representations of different embodiments of molds that can be inserted into individual wells of a microtiter plate;

FIG. 10 shows a special embodiment of the system according to the invention, in which a suction system and a collection compartment are integrated.

The present invention uses, for the solution of the above-mentioned problem, in each case an opening in an inlay, preferably a compensation capillary tube, which is not in direct contact with the receiving cavity in a well of the partially active micro fluidic system (microtiter plate). The inlay forms a cavity within the well for the reception of a cell culture. By means of the capillary openings, a pressure compensation is achieved. In addition, the openings are used as a filling guide for the capillary tips of pipetting systems, preferably of pipetting robots, which can simulate active throughflow through such a system by the periodic uptake and release of reagent.

FIG. 1 shows in a simplified representation a first embodiment of a partially active microfluidic system for 3D cell cultivation. Through the capillary openings 1 it is possible to introduce fluid, for example, by means of pipetting tips 2. The capillary openings 1 moreover already allow the obtention of inlays 3 that are colonized by cells, and provide a porous 3D culture carrier, without introducing the risk of floating into a microtiter plate 4 filled with fluid.

In FIG. 2, a second embodiment is represented, which provides a solution for cocultivation problems. According to the invention, it is also possible here to introduce several inlays 3, 3a into the microtiter plate 4. Here, only the uppermost inlay 3 has the openings 1. By means of this special embodiment, exposure to flow can be achieved for adhering cells, or, a reaction volume can be generated for free floating cells.

According to the modified embodiment shown in FIG. 3, it is also possible for all the inlays 3, 3a that are integrated in the microtiter plate 4 to present the openings 1, which are interconnected in each case. As a result, floating of the cells in the lower area of the microtiter plate well can be prevented.

FIG. 4 is a representation of the principle of the method according to the invention. Cyclic addition by pipetting through the pipetting tips 2, for example, into a capillary 5 leading onto the well bottom, and removal via additional pipetting tips 6, which, for example, are immersed only above the uppermost inlay 3, generates a pulsating fluid stream and simulates a pump system. In this way, one achieves that the fluid continues to flow through the porous 3D cell culture carrier, and the supply of the cells is ensured. The pipetting cycle can also occur in the reversed direction.

FIGS. 5-9 show detail representations of different embodiments of molds 10 which form inlays 3. The formed cavities are introduced into the individual wells of the microtiter plate, which is not shown here. The mold 10 can be shaped so it has different depths and different diameters. The mold must provide sufficient space for 3D cell cultivation and sufficient medium in the same compartment. If the mold is to be suspended in a well of a 96-well microtiter plate, it has preferably a diameter that is slightly less than that of the well. If several molds 10 are stacked (see below), then they can be shaped differently to improve the stackability (for example, with decreasing diameter).

According to FIG. 5, the mold 10 first forms the cavity or a sample compartment 11. The bottom side of the sample compartment 11 is closed by a porous sample support 12 to which the cell culture is applied. The sidewalls of the mold 10, depending on the application, present either a porous design (undirected substance transport occurs) or a nonporous design (for directed substance transport).

The sample support 12 and/or sidewalls of the mold possess a two dimensionally or three dimensionally structured sieve structure, which is preferably restricted to the surface in the bottom area. It is decisive that the mold 10 to be suspended represents at its lowest place (here sample support 12) not only a sieve structure (with a pore diameter that is smaller than the cell diameter, preferably less than 5 μm); rather it must also be structured in a way that is appropriate for cell culture, preferably three dimensionally (for example, with recesses, in the form of a foam, and the like), because the mold is used for the cultivation of cells, and in many cases 3D structuring offers advantages in maintaining or generating cell differentiation. 2D structuring could consist of a physical or chemical modification/patterning, in such a way that the cells adhere/stick better, more poorly, or regionally differently, depending on the application.

The mentioned sieve structure here means that, although the pores have to be smaller than the cell diameter, they must also be large enough to allow the flow of medium from compartment to compartment. For example, if one removes the fluid from a mold 10, which is immersed in such a way in the fluid and fixed therein that now the fluid level is higher than the bottom of the suspended mold, then the fluid should, due to the hydrostatic pressure, and in part also due to capillary forces, flow through the sieve structure in the bottom of the mold 10 into the compartment 11 which is delimited by the mold. The same can be achieved if one does not first remove the fluid in the mold, and, instead, builds up a hydrostatic pressure in the surrounding fluid reservoir by fluid addition. For this purpose, the design can also be such that the volume of the surrounding fluid reservoir is greater than the volume of the sample compartment. However, the sieve structure serves not only to allow throughflow, but also as a cell culture substrate/sample support, preferably as a 3D cell culture substrate. Thus, one produces not only a simple membrane, but at least a 3D structured membrane or a porous foam or similar material.

As can be seen in FIG. 6, a pipetting section 13 is formed preferably on the mold 10, which section has the opening 1 into which a medium can be introduced through the pipetting tip 2. When the pipetting section opens above the sample support 12, a flow direction of the medium—symbolized by the flow arrow 14—is generated when medium is added, and results in a perfusion of the cell culture.

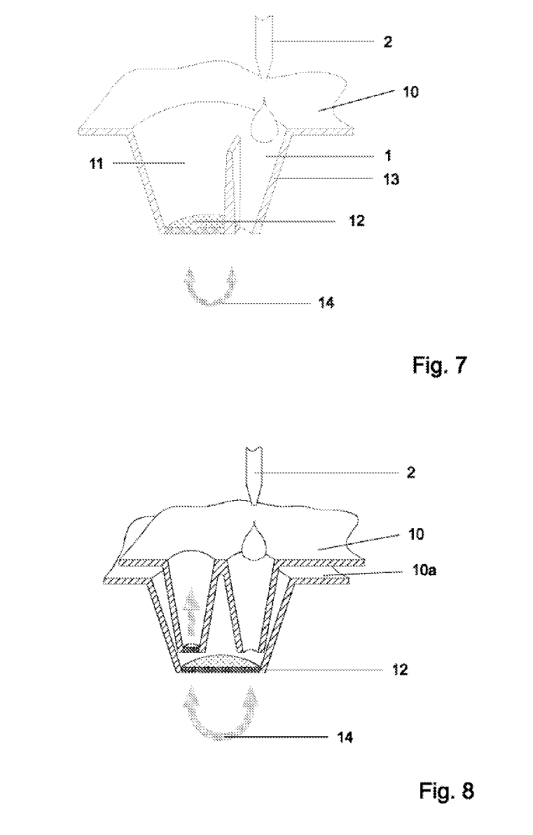

FIG. 7 shows a modified embodiment, in which the pipetting section opens approximately in the plane of the sample support 12. Depending on the addition or removal of medium, one can change the flow direction of the medium via the set fluid level in the pipetting section.

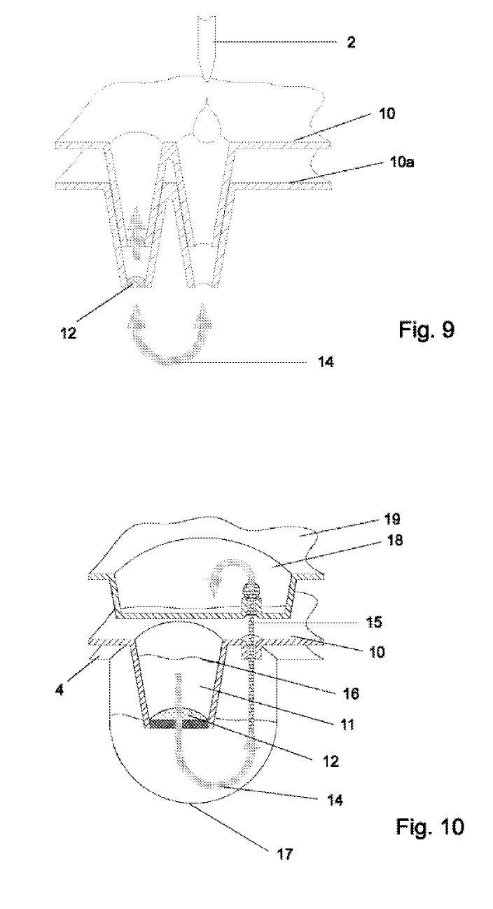

FIGS. 8 and 9 show variants for stacking several molds 10, 10a. This allows several planes of cell culture, which increases the range of application of the object of the invention (for example, different cell types on each culture plane).

FIG. 10 represent a special embodiment of the system, in which an integration of a suction system and of a collection compartment is carried out. The suction system here consists of a fibrous or porous material, which is characterized by very high capillary forces and forms a wick 15. If a high level 16 of medium is now used in the large size sample compartment 11, and the wick 15 is immersed until it reaches the medium located in the surrounding well 17 (or another fluid container), the high capillary forces in the suction system 15 convey the medium into a collection compartment 18 of a second mold 19. This effect can be reinforced by filling the collection compartment 18 with a strongly absorbing material. As a result of the dimensioning of the suction system 15, one can set the desired conveyance performance. Thus, throughflow through the sample support 12 can be achieved for a longer duration without any auxiliary means or active components. This active throughflow leads necessarily to a better supply of the cells. Thus, this embodiment offers an additional significant advantage compared to known systems.

The partially active microfluidic system according to the invention can be produced in all volumes and size ranges that can be implemented with pipetting robots.

### LIST OF REFERENCE NUMERALS

1 Openings, preferably capillary openings
2 Pipetting tips for addition by pipetting
3, 3a Inlay
4 Microtiter plate
5 Capillaries
6 Pipetting tips for removal by suction
10, 10a Mold
11 Sample compartment
12 Sample support
13 Pipetting section
14 Flow arrow
15 Wick/suction system
16 Level in the sample compartment
17 Well
18 Sample compartment
19 Second mold

1. A partially active microfluidic system for cell cultivation comprising a multiwell system with several wells with cell receiving cavities characterized in that:
(a) each cavity comprises (i) at least one inlay having an opening that does not directly open into the cavity and does open into a space surrounding the cavity, and (ii) a medium; and
(b) any medium present in the surrounding space communicates via a sieve structure in the at least one inlay with the medium contained in the cavity.

2. A partially active microfluidic system according to claim 1, wherein the inlay is designed as a two-dimensional or three-dimensional support structure for three-dimensional cultivation of cells.

3. A partially active microfluidic system according to claim 1, wherein the openings comprise capillary openings on the inlay.

4. A partially active microfluidic system according to claim 1, wherein the openings are arranged in pipetting sections which pipetting sections are separate from the cavities.

5. A partially active microfluidic system according to claim 1, wherein the inlays comprise molds having at least one sample compartment as a cavity, and wherein at least the bottom of the sample compartment comprises a sample support with a sieve structure.

6. A partially active microfluidic system according to claim 5, wherein the molds comprise sections having a two-dimensionally or three-dimensionally structured sieve structure.

7. A partially active microfluidic system according to claim 5 wherein the molds are suspended in a microtiter plate (MTP) and closed with a cover.

8. A partially active microfluidic system according to claim 1, comprising several inlays stacked on top of each other, wherein at least the uppermost inlay comprises openings.

9. A partially active microfluidic system according to claim 8, wherein all the inlays comprise openings and the openings of the individual inlays are connected to each other.

10. A method for the perfusion of a partially active microfluidic system according to claim 1, wherein fluid is pipetted by means of a pipetting tool into the openings of the inlay(s) and removed above the inlay(s).
